(19) United States
(12) Patent Application Publication
(10) Pub. No.: US 2023/0355869 A1
(43) Pub. Date: Nov. 9, 2023

(54) Wearable Drug Infusion Device

(71) Applicant: SFC Fluidics, Inc., Fayetteville, AR (US)

(72) Inventors: Forrest W. Payne, Fayetteville, AR (US); Bradley Thomas Ledden, Fayetteville, AR (US); Emma Qian Sun, Fayetteville, AR (US); Anna M. Washburn, Lillian, AL (US); Barry H. Ginsberg, Arlington, VA (US)

(21) Appl. No.: 18/028,442

(22) PCT Filed: Oct. 1, 2021
(86) PCT No.: PCT/US2021/053137

§ 371 Date: Mar. 24, 2023

Related U.S. Application Data
(60) Provisional application No. 63/086,663, filed on Oct. 2, 2020.
(60) Provisional application No. 63/191,155, filed on May 20, 2021.

(51) Int. Cl. A61M 5/142; A61M 5/168; A61M 5/14

(52) U.S. Cl. CPC A61M 5/14244; A61M 5/16813; A61M 5/16886; A61M 5/1408; A61M 2205/3334

(57) ABSTRACT

A low dose drug delivery pod comprised of a reciprocating pump, an active valve set, and, in some embodiments, a dispense confirmation sensor. The active valve set can be designed with mechanical interference between them so that both the inlet and outlet valve must close before either one opens, ensuring that at least one of the two valves is closed at all times. The dispense confirmation sensor is a flow-based detection system that provides rapid feedback if an individual dose has or has not been delivered.

### CROSS-REFERENCE TO RELATED APPLICATIONS

This application claims the benefit of U.S. provisional patent applications No. 63/086,663, filed on Oct. 2, 2020, and No. 63/191,155, filed on May 20, 2021. Such applications are incorporated herein by reference in their entirety.

### STATEMENT REGARDING FEDERALLY SPONSORED RESEARCH OR DEVELOPMENT

This invention was made with government support under grant nos. R43DA041173, R44DA0411173, R43DK110972 and R44DK110972 awarded by the National Institutes of Health. The government has certain rights in the invention.

### BACKGROUND OF THE INVENTION

This invention relates to wearable drug infusion devices.

Although a number of pharmaceuticals can be delivered via wearable pods, many of the products and the intellectual property covering drug infusion pods are used for insulin delivery. United States patents that describe insulin pumps include U.S. Pat. Nos. 9,717,849; 9,841,830; 10,279,106; 10,413,682; 10,549,030; 10,646,652; 10,695,492; 10,933,188; 10,967,137; 11,033,677.

Insulin pump therapy has led to dramatic improvements in the treatment of diabetes over the last forty years. Despite the clinical benefits of an insulin pump, the adoption rate of such devices has remained relatively low in the US with only about 35% of patients with Type 1 diabetes and fewer than 5% of patients with Type 2 diabetes using them. This low adoption is primarily due to product design shortcomings of current devices. For instance, even with today's insulin pumps, errors of insulin infusion can occur due to pump failure, insulin infusion set (IIS) blockage, infusion site problems, insulin stability issues, user error, or a combination of these. Collectively, insulin pumps and their components are responsible for the highest overall number of malfunctions, injury, and death reports in the FDA's medical device database, according to an Associated Press analysis of reports since 2008.

Even though overdose of insulin can be dangerous to many insulin pod users, most pods either have no valves or rely on passive valves such as duckbill valves to control flow from the pump. Flow can be generated in a pumping system by any differential pressure, be it a small leak in a fluid path, a rise or drop in atmospheric pressure surrounding the pod, or a patient applying pressure to the pod area for example, by laying on it. Passive valves are a simple option in reciprocating microfluidic systems that rely on channel structure or natural effect to regulate flow rate. In the case of duckbill, umbrella, or other passive valve types, flow is theoretically only allowed in one direction (toward the patient) and the valve is opened passively at an engineered cracking pressure by the fluid flow generated by the pump. These passive valves can be opened by flow for any reason, including direct force on the pod or by air pressure changes such as those that can occur during flight. Additionally, passive valves do allow small amounts of backflow that can be a significant portion of a user's insulin needs, especially at basal delivery rates for low body mass individuals. In these cases, the patient is exposed to the possibility that an under or overdose of insulin is delivered from the reservoir to the patient. In other words, passive valves allow no mechanism to accurately meter a dose to the patient. Additionally passive valves do not allow for a dual-sided pump to be used to independently deliver two drugs using a single pumping mechanism.

Additional design shortcomings in pressure-based occlusion sensors commonly used in modern drug delivery pods also expose users to significant and potentially fatal hazards. Interruption of insulin infusion can result in hyperglycemia, ketosis, and ketoacidosis. Conversely, delivery of excessive insulin can cause severe hypoglycemia, which can be deadly. When insulin infusion from a pump is blocked or stopped for any reason, the user must be warned as soon as possible because ketosis and hyperglycemia/ketoacidosis can rapidly develop. In currently available insulin delivery pods, pressure sensors are used to detect occlusions. In this case, the pressure generated from the pump must reach a threshold limit in order to activate an occlusion alarm. At low basal rates, these sensors can take many hours to alarm. For other no-flow conditions, such as a leaking connection in the pump or other pump failure, pressure-based sensors are completely ineffective. Limitations in occlusion detection result in adverse clinical events, such as unexplained hyperglycemia that is not preceded by an occlusion alarm. A 2011 study concluded that about 65% of participants (n=256) in a controlled trial of pump therapy experienced at least one episode of unexplained hyperglycemia per month. These unexplained hyperglycemia events can result in a patient stopping insulin pod therapy altogether.

Researchers have observed that pressure excursions high enough to reduce the rate of insulin flow seldom surpassed the pressure thresholds of occlusion sensors, giving rise to sub-alarm, or silent, occlusions associated with hyperglycemia. The detection of occlusions in insulin infusion sets is an important safety feature. However, using certain current pods, an occlusion alarm generally takes 2 to 4 hours after an occlusion at the standard basal rates (the occlusion detection time). For most pumps the occlusion detection time is volume-dependent, so the occlusion detection time for a basal rate such as 0.5 U/h is approximately double that of a basal rate of 1.0 U/hr.

Currently available insulin pods have problems that make them less applicable for use with low body mass individuals with diabetes, such as children, adolescents, and many young adults. Perhaps the most important is low accuracy at reduced basal rates. Due to mechanical limitations, currently marketed pods that are precise at standard dose basal rates (1.0 U/hour), exhibit a significant degradation in their precision for the low basal rates needed by patients with low body weight. The calculation of Total Daily Dose (TDD) of insulin is different for children, adolescents and adults but ranges from about 10 U/day for a 6-year-old to 60 U/day for an 18-25-year-old adult. Therefore, the basal rates (about 50% of TDD) needed for some of these patients are very low, and they depend on the ability to program these low rates and have them delivered with accuracy. For example, the lowest basal increment of the Omnipod® from Insulet Corporation is reported to be 0.05 U, so if an increment is given every 6 minutes (as is standard) this equates to a basal rate of 0.5 U/hour, which is applicable for an approximately eighty-pound person. If the patient weighs less than eighty pounds then the basal dose must be given less frequently than every 6 minutes, setting up a situation where missed doses and dosing inaccuracies become more clinically significant.

With insulin-only pods, the benefits of tight glycemic control for reducing microvascular complications are hindered by an increased risk of hypoglycemia, and that fear of hypoglycemia often leads to permissive hyperglycemia. The need for a diabetes management system that allows tighter glycemic control in patients with Type 1 diabetes while significantly reducing incidence of hypoglycemia remains as critical as ever. According to the American Diabetes Association and the International Diabetes Foundation, there are currently four million people with Type 1 diabetes (T1D) in the US, EU, and Japan alone. Furthermore, the incidence of T1D is on the rise. It has been reported that if incidence rates continue to increase on their existing path, global incidence could double over the next decade. As many as 50% of patients with T1D are first diagnosed as children in the age range of 0-4 years, and it is recommended that intensive management be initiated upon diagnosis. Teens also represent a difficult group to manage. It can be expected that up to 40% of teenagers may have a period of pervasive non-compliance with diabetic routines; currently, a child diagnosed with T1D at age ten will, on average, lose about nineteen years of life as a result of the disease.

Another patient subset that is greatly affected by ketoacidosis resulting from silent occlusions are pregnant women. Pregnancy, with major changes in hormonal status, makes insulin control more difficult in the many patients with diabetes. Pregnancy predisposes a patient with diabetes to hyperglycemia and ketoacidosis due in part to increased insulin resistance and accelerated cell starvation, especially during the second and third trimesters. In the mother, ketoacidosis can lead to uncontrolled hyperglycemia, dehydration, loss of electrolytes, ketosis, and metabolic acidosis. It is considered a medical emergency that, if left unchecked, can lead to maternal complications such as renal failure, cerebral edema, coma, and death. Impacts on the fetus can be significant and may happen at minimal levels of ketosis. They include fetal brain injury, long-term developmental impacts, and death. In one study, among 77 ketoacidosis events in 64 pregnancies the following occurred: fetal demise 16%, preterm birth 46%, and NICU admission 59%. When fetal demise occurred, 60% of the deaths were within one week of the ketoacidosis event. Ketoacidosis occurs during diabetic pregnancy of women with T1D, T2D, and gestational diabetes at rates as high as 10%.

Insulin-only pods are not able to maintain adequate glycemic control in many patients with T1D and are wholly unable to correct a low blood glucose condition. There are currently no commercially available dual-hormone pods capable of delivering both insulin and glucagon. Some research has been done on the benefits of dual-hormone delivery using two t:slim pumps (one for insulin and one for glucagon), where it was reported that, as compared with an insulin pump, a wearable, automated, bi-hormonal, bionic pancreas improved mean glycemic levels, with less frequent hypoglycemic episodes among both adults and adolescents with T1D.

Mechanically-based miniaturized syringe pump technology is currently used as the pumping mechanism for the vast majority of commercially available drug-delivery pods. One important issue with syringe pumps is the common occurrence of significant over and under dispense volumes due to friction and stiction forces between the adjacent sliding and stationary parts of the pumping mechanism. For insulin pumps, the accuracy data that has been accepted for decades is provided in trumpet curves that represent an increasing time average of dispense volume errors over a twenty-four-hour window. Trumpet curves show the precision of the dosing and the long-term accuracy in a single graph. The over and under dispenses are less clinically relevant for diabetic patients that weigh over eighty pounds but can be harmful or even dangerous for lower body weight patients. Additionally, low body weight patients typically require smaller dose volumes, and syringe pump-based pods are less accurate at smaller dose volumes due to stiction and inherent limitations in stepper action.

Modern insulin therapy using pumps requires administering small doses of insulin every few minutes. Small adults and adolescents may require doses as small as 0.05 U of U100 insulin (500 nL) every six minutes and these tiny doses need to be delivered with accuracy and precision. Furthermore, the potential use of concentrated insulin U200, U300 or U500 reduces the pumped volume further by 50%, 67% and 80%, respectively, making accuracy and precision of very small doses even more critical. These concentrated insulins are expected to allow pumps to be smaller to increase adoption in the future, but only if they can be pumped accurately. An additional upcoming challenge is that artificial pancreas devices of the near future will make alterations in dose volume every five minutes and will require flexible resolution of dose delivery.

A connected automated insulin delivery (AID) system is an insulin delivery pod in communication with a continuous glucose monitor and a dosing control algorithm that minimizes user input and automates insulin delivery as much as possible. In an AID system, there is clear recognition that errors from one device can be propagated throughout the system, potentially resulting in failure of the entire system. One of the major concerns in an AID is the failure of recommended insulin delivery and how the system will respond to that partial or complete failure. A full or partial occlusion may result in the algorithm overcompensating for insulin that it thinks has been delivered, but, in fact, has not. Rapid pump failure detection would help to prevent an additive effect from occurring due to improper data being applied in the algorithm.

An occlusion of an insulin infusion set interferes with glycemic control and reduces confidence in the use of insulin pumps. Prolonged or recurrent hyperglycemia caused by occlusions can result in long-term complications like retinopathy and neuropathy. A more effective, instantaneous occlusion sensor could detect life-threatening blockages in the system and alert the user in time to take appropriate steps to correct the problem and minimize negative effects.

References mentioned in this background section are not admitted to be prior art with respect to the present invention. All references cited are incorporated by reference as if fully set forth herein.

### BRIEF SUMMARY OF THE INVENTION

The present invention is directed to a drug delivery pod with dispense confirmation realized using miniaturized reciprocating pumps rather than syringe pumps. Appropriate pumps used in various embodiments may include, but are not limited to, electrochemical pumps; electrochemiosmotic pumps such as described in U.S. Pat. Nos. 7,718,047, 8,187,441, and 8,343,324; and electroosmotic pumps such as described in U.S. Pat. Nos. 9,745,971, 10,156,227, and 11,015,583. The present invention incorporates active safety valves, with actuation mechanisms in various embodiments that may include, but are not limited to, magnetic; electrostatic; thermal; phase change; piezoelectric; or rheological or externally provided forces. The valve mechanism in various embodiments may incorporate dual-latching microvalves such as disclosed in U.S. Pat. No. 10,576,201. In various embodiments the invention may also incorporate rapid dispense indicators, including but not limited to optical detectors, thermal time-of-flight, and electrochemical detectors such as described in U.S. Pat. No. 10,690,528.

In various embodiments of the present invention, accuracy at low doses and safety of the drug delivery system overall is enhanced by the addition of active safety valves sets that control fluid flow at two locations in the fluid path. One active valve, the inlet valve, is located between the drug reservoir and the reciprocating pump, while the second active valve, the outlet valve, is located between the reciprocating pump and the patient. The two valves are operated as a set to allow a measured dose volume into the dosing chamber between the two valves, and then to allow dispense of that measured dose to the patient. In some embodiments, these valves may be designed with mechanical interference between them so that both the inlet and outlet valve must close before either one opens, ensuring that at least one of the two valves is closed at all times. There is never an open path between the reservoir and the patient, which is an important and unique safety feature.

Another enabling safety feature in various embodiments of the present invention is the dispense confirmation sensor (DCS), which is a flow-based detection system that is operated during dispense to ensure that the system is operating as expected and provides immediate feedback that an individual dose has or has not been delivered. Most currently available pods rely on pressure-based sensors, which have limited ability to provide information on a single dispense. Indeed, many of the pressure-based occlusion sensors that are currently in use do not notify the user until hours after an occlusion has occurred, if at all. Additionally, pressure-based sensors only notify of an occlusion, whereas the DCS that may be incorporated into the present invention can notify of a single missed dispense that occurs for any reason, including empty reservoir, pump failure, or occlusion. Since children and adolescents are more likely to require a lower basal insulin rate, they are in more danger from silent occlusions and from long occlusion detection times. If lack of insulin delivery goes unnoticed, the patient is increasingly at risk of hyperglycemia and ketoacidosis. Even non-emergency high blood glucose excursions are of substantial concern during adolescence because the maintenance of near-normal blood glucose levels is needed to ensure normal development and to avoid long-term health effects. Because the DCS does not rely on the buildup of pressure to alarm an occlusion, the present invention in various embodiments identifies an occlusion (or other device failure) immediately and alerts the user within a clinically relevant timeframe even at the very low basal delivery rates required by low body weight individuals.

The combination of the reciprocating pump (such as an electrochemiosmotic pump or “ePump”), safety valve sets, and dispense confirmation sensor (DCS) collectively confer unique and superior operation to the present invention. In addition, in certain embodiments the present invention has been engineered to minimize its size and power requirements to enhance usability and lower overall cost to the consumer.

These and other features, objects and advantages of the present invention will become better understood from a consideration of the following detailed description of the preferred embodiments and appended claims in conjunction with the drawings as described following:

### BRIEF DESCRIPTION OF THE SEVERAL VIEWS OF THE DRAWINGS

FIG. 1 is a diagram showing a procedure for dispensing insulin in a first embodiment of the present invention.

FIG. 2 is a partial cut-away elevational view of a first embodiment of the present invention.

FIG. 3 is a chart showing a comparison of accuracy data between commercial devices and a first embodiment of the present invention.

FIG. 4 is a chart showing dosing resolution of a first embodiment of the present invention.

FIG. 5 is a diagram showing a procedure for dispensing insulin without glucagon in a second embodiment of the present invention.

FIG. 6 is a diagram showing a procedure for dispensing glucagon without insulin in a second embodiment of the present invention.

FIG. 7 is a partial cut-away elevational view of a second embodiment of the present invention.

FIG. 8 is a chart showing insulin dispense (bottom graph) and glucagon dispense (top graph) over a 48-hour time period according to a second embodiment of the present invention.

FIG. 9 is a chart showing trumpet curves for dosing error of both insulin and glucagon, separately according to a second embodiment of the present invention.

FIG. 10 is a chart showing dispense accuracy for a second embodiment of the present invention.

FIG. 11A is a chart showing response of DCS (μA) for an 8 μl insulin dispense versus an occlusion in a first embodiment of the present invention.

FIG. 11B is a chart showing response of DCS (μA) for an 8 μl glucagon dispense versus an occlusion in a second embodiment of the present invention.

FIG. 12A is a chart showing response of DCS (μA) for a 0.5 μl insulin dispense versus an occlusion in a first embodiment of the present invention.

FIG. 12B is a chart showing response of DCS (μA) for a 0.5 μl glucagon dispense versus an occlusion in a second embodiment of the present invention.

FIG. 13 is a chart showing blood glucose over time in an in vivo test comparison between a syringe pump and the second embodiment of the present invention.

### DETAILED DESCRIPTION OF THE PREFERRED EMBODIMENT(S)

Before the present invention is described in further detail, it should be understood that the invention is not limited to the particular embodiments described, and that the terms used in describing the particular embodiments are for the purpose of describing those particular embodiments only, and are not intended to be limiting, since the scope of the present invention will be limited only by the claims.

In an embodiment of the present invention, both sides of a reciprocating pump with two sets of safety valves and two dispense confirmation sensors (DCSs) allows for superior control of the independent delivery of two therapeutic fluids from a single pod. One may imagine many situations where automated delivery of two therapeutic fluids would be optimal, including antibiotics and pain killers and timing of multiple therapeutic fluids to treat cancer, HIV and mental/emotional disorders. The following sections focus on one very important therapeutic fluid pairing: the blood glucose counterbalancing hormones, insulin and glucagon. It should be noted, however, that the invention is not limited to this example application.

For normal glucose homeostasis, humans have bi-directional hormonal systems, with negative feedback control. Insulin, produced by the pancreatic beta cells, lowers glucose, and four hormones raise blood glucose concentration. The blood glucose is raised by the fast onset hormones, glucagon and epinephrine, and by the slower, longer-lasting hormones, cortisol and somatotropin. Bidirectional homeostatic systems are also present for calcium metabolism and for control of some T-cells. Bidirectional systems are almost always more stable. Preliminary studies using two pumps to deliver insulin and glucagon under algorithmic control have shown increased time in range and reduced time in hypoglycemia. There are several subpopulations of insulin-dependent patients with diabetes mellitus that have been clearly shown to benefit from the addition of glucagon to an automated drug delivery system, including the hypoglycemic unaware, low body weight patients, patients who experience frequent hypoglycemia and patients who want to exercise.

It is reported that about 17% of people with T1D are hypoglycemic unaware, meaning that these patients do not feel any symptoms when their blood sugar levels become dangerously low. This is very dangerous because low blood sugar can lead to unconsciousness, seizures, or other conditions that prevent the patient from correcting the low glucose condition, which can result in death. This condition is prevalent in children and in patients who have repeated hypoglycemic events—so having the condition tends to make the condition worse. This population will clearly benefit from having an automated glucagon source at the ready to deliver glucagon in response to an existing or predicted low blood sugar event.

Patients with Type 1 diabetes have increased risk of hypoglycemia during exercise caused by a combination of acutely increased glucose sensitivity, insulin absorption and glucose uptake. Fear of hypoglycemia is a mental barrier to exercise among the population that has T1D, and it has been reported that greater than 60% of adults with T1D are sedentary. A bi-hormonal artificial pancreas may lead to an increased quality of life for this population by reducing the fear of exercise-induced hypoglycemia. Insulin-only systems that attempt to correct for exercise must be notified of an expected exercise period as long as 90 minutes prior to the onset of the exercise. In addition to not always being able to anticipate exercise, the duration and intensity of an upcoming exercise session may be difficult to estimate. This often results in patients with T1D avoiding exercise altogether, which reduces overall health.

Other known target outcomes for bi-hormonal control of a drug delivery system include reduction in carbohydrate intake, reduction of mean glucose levels, tighter glycemic control throughout the day and night, and reduction of severe hypoglycemia. One embodiment of the present invention is a low-profile pod that allows for bi-hormonal therapy in a single pod. A purpose of this design is to reduce the mental and body burden that currently prevents the realization of practical bi-hormonal glycemic control.

Regular physical activity has numerous benefits for people with T1D but also complicates blood glucose control. Factors that affect blood glucose concentration during physical activity include activity type, intensity and duration, as well as the amount of insulin and food in the body; all which complicate the goal of balancing the rate of glucose release and uptake in the bloodstream. During a clinical trial of a multivariable insulin-only artificial pancreas, it was found that blood glucose response varied with moderate continuous training (with a trend toward hypoglycemia) as compared to high-intensity interval training (with a trend toward hyperglycemia) and that blood glucose needed to be managed closely for up to eleven hours after exercise. Currently, the two methods to combat hypoglycemia following exercise are reduction of insulin prior to exercise (which requires planning of physical activity) and consumption of additional carbohydrates (which is not optimal if the patient is trying to lose weight).

Due to the limitations of insulin-only pods in normalizing blood sugar during and after exercise, many researchers began to look into the benefits of the addition of automated glucagon delivery to such systems. One group found that dual-hormone pods reduced the risk of hypoglycemic events overall and during exercise. The researchers completed a four-day outpatient study with four arms: dual-hormone, single-hormone, predictive low glucose suspend, and continuation of current care. Each four-day arm included three moderate intensity exercise sessions. Twenty adults completed each arm of the study. For the dual hormone arm a modified fading memory algorithm was used for dose control of insulin aspartate and GlucaGen® glucagon reconstituted every 24 hours. Two t:slim (Tandem Diabetes Care) pumps were used, one for each hormone. Of all four arms, the dual-hormone tests showed time in hypoglycemia was lowest both during exercise and overall and also required the lowest number of carbohydrate consumption treatments to raise blood sugar. The researchers stressed that automated hormone delivery is especially important for patients who forget to adjust prior to exercise or when exercise in unplanned. They also recommended that to avoid depletion of hepatic glycogen that the glucagon dose should be kept as low as possible. The researchers showed that using glucagon during exercise can help reduce the occurrence of hypoglycemic events caused by exercise. These events can even occur hours after the exercise has been completed due to increased glucose uptake in the muscles during the healing and recovery phase.

Another study examined the benefits of automated dual-hormone delivery in a randomized crossover trial comparing single-hormone artificial pancreas, dual-hormone artificial pancreas and continuous subcutaneous insulin infusion (CSII) for twenty-four hours including an exercise session. Both types of artificial pancreases excelled over traditional CSII therapy. However, the dual-hormone artificial pancreas showed significant advantages over single-hormone artificial pancreas, including a 50% reduction of time in hypoglycemia, with special benefits in reducing overnight hypoglycemia following evening exercise. Dual-hormone artificial pancreas also reduced the need for carbohydrate consumption to treat hypoglycemia by five times as compared to single-hormone artificial pancreas.

Current automated pumps for treatment of diabetes are single-hormone devices, delivering only insulin. Hypoglycemia, although decreased in frequency with the use of these devices, occurs and must be treated with oral glucose or exogenous glucagon. Several devices are being developed that utilize both insulin to lower glucose and glucagon to raise the blood glucose levels and rescue from hypoglycemia. These devices can utilize glucagon to prevent or correct a hypoglycemic condition caused by either insulin overdosing and/or exercise. In these devices, the glucagon can also be judiciously administered to carefully titrate blood glucose into the mid-normal range. These correction doses are likely to be in to 25 to 50 μL range (100-200 μg of 4 mg/mL glucagon), so accuracy and precision of delivery will still be important to prevent over-correction, resulting in more insulin needing to be delivered.

There are several ways in which glucagon can be incorporated into an artificial pancreas: 1) as a rescue hormone, used to raise a hypoglycemia blood glucose level up to the normal or higher level; 2) to prevent hypoglycemia based upon the rate of change of a descending blood glucose level; 3) to prevent hypoglycemia due to exercise; and 4) integrated into the algorithm to maximize centricity around the preferred glucose level. Using glucagon in an algorithm may be complex. Although the glucose response to glucagon is approximately linear over the range of 25-250 μg (corresponding to volumes of 6-63 μL for stable glucagon formulations), the response is highly dependent on the ambient insulin concentration. When algorithmically using glucagon to prevent hypoglycemia based upon model projections of glucose or to prevent the hypoglycemia of exercise, accuracy is important. When using glucagon as part of bi-hormonal homeostatic control, accuracy is critical.

In certain embodiments, the present invention is extremely accurate for very low pumped volumes, with the ability to accurately deliver volumes that would correspond to glucagon induced glucose changes of 1 mg/dL or less. The ability to deliver any dose (as opposed to the discreet volumes of other pumps) of glucagon and the ability to determine that the dose was delivered allows the algorithm great flexibility to keep the blood glucose in the target range. The ability to perform this operation in a small single-pump pod that is only larger than an insulin-only pod by the size of the glucagon reservoir is more discrete and has a smaller on-body burden than a durable pump or two pods.

Additionally, as sensor augmented pumps are developed to the point that they also incorporate automated insulin dosing and dose recommendations using an algorithm, it is important for the algorithm to be able to verify that the doses are being given as recommended. It is very difficult to design an algorithm that can discern several missed insulin doses from a blood glucose value that is still rising after a dose has been given. In the short term, an algorithm would over-correct for a missed dose in its calculations, calculating that sufficient insulin has not been given to correct the rise in blood glucose without knowing the cause. In the worst case, if the occlusion, be it a kink or other temporary blockage, is released, then excessive insulin would be delivered upon release of the occlusion, which could rapidly lead to hypoglycemia. In these cases, it would be helpful that a computational algorithm is informed by the Dispense Confirmation Sensor (DCS) that a dose(s) was not administered correctly so that the algorithm could take the missed dose(s) into account when administering future doses or deciding when to trigger an alarm and which alarm to trigger. Because glucagon is used to raise blood sugar in order to prevent or treat hypoglycemia, failure of a glucagon dispense could have life threatening consequences, especially if a user is hypoglycemic unaware or if hypoglycemia occurs when the patient is asleep. The DCS has multiple benefits above and beyond current occlusion sensors that greatly improves safety and efficacy of insulin/glucagon delivery, especially for high-risk patients. These benefits include:
Confirmation of single dose dispensing down to a single basal dispense;
Speed of dispense detection allows for confirmation of dispense or dispense error prior to the next dispense;
Time to alarm of missed dispense based on clinical relevance;
Physician can fine-tune alarm settings to best meet the needs of each patient;
Occlusion detection can be subdivided into either “alert” or “alarm” conditions;
Missed dispenses will be alarmed for any no-flow condition (not just occlusion);
Dosing algorithms will be better informed of actual dosing in real-time;
End user patients will have improved confidence in device performance; and
Benefits of DCS are realized in a very small footprint, with minimal power requirements and low cost of goods.

The combination of instantaneous occlusion detection and higher resolution and precision of dose volume provides significant advantages over existing drug delivery pods. Furthermore, the ability of the second side of the reciprocating pump to deliver glucagon as needed to prevent or correct a hypoglycemic condition provides for more effective treatment of Type 1 diabetes by preventing or correcting hypoglycemic conditions due to exercise or over-bolusing of insulin through accurate automated delivery of glucagon with rapid dispense confirmation; preventing hyperglycemic conditions through more accurate dosing of insulin and faster detection and acknowledgement of conditions in which insulin is not being delivered for any reason; and maximizing centricity of time in range with algorithmic control.

The incorporation of these features into various embodiments of the present invention is in the best interest of patients, as they improve the standard of care and lead to a reduction in serious adverse events, including some higher-risk user populations such as hypoglycemic unaware, low body weight, or pregnant patients, and will allow more freedom for patients who want to exercise.

The DCS is particularly important for a dual-hormone pump. Some artificial pancreas algorithms take advantage of the ability of glucagon to rescue a patient from more aggressive insulin usage, by either giving a large dose of glucagon in the presence of hypoglycemia or to alter the slope of a glucose pattern predicted to lead to hypoglycemia. The DCS can detect the failure of delivery, even if the failure is partial and warn the patient. This happens within seconds of the delivery failure and is an important safety feature that allows for notification of dispense error prior to the next dispense. Additionally, the ability of the DCS to operate with liquid glucagon formulations permits immediate alert/alarm based on clinical significance. In the instance of missed glucagon dispense with predicted hypoglycemia an alert may be issued to notify the patient. However, missed glucagon dispense during hypoglycemia may result in an alarm which will indicate that the patient must correct the condition. As dual-hormone algorithms are developed, they may utilize the potential for negative feedback to more aggressively maintain a tighter blood glucose range. The rapid response of the DCS is an important safety feature for these aggressive algorithms.

The present invention in various embodiments provides the previously unavailable ability to prevent or correct a hypoglycemic condition by delivering glucagon in response to a falling blood glucose level. It also provides much better accuracy at low dose volume and far superior occlusion detection, especially at the low flow rates, as compared to prior art pods. All of this in a pod that is only larger than an insulin-only pod by the size of the glucagon reservoir. The dual-hormone version of the present invention provides patients a safer automated hormone delivery experience than what currently exists on the market and contributes to better glycemic control and improved health outcomes for insulin-dependent patients with diabetes. The combination of superior precision of dose delivery and real-time dispense confirmation offers significant advantages over existing alternatives and is in the best interest of patients suffering from this debilitating human disease.

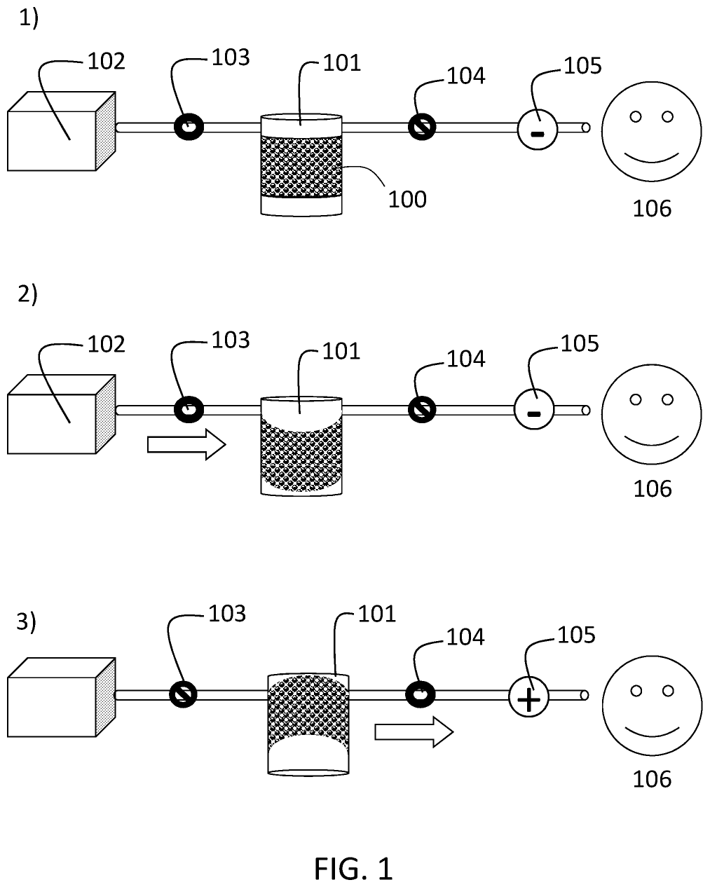

Embodiments of the invention will be described herein both for delivery of insulin only and for delivery of both insulin and glucagon. (As already noted, the invention is not limited to these particular therapeutic fluids, and others may be substituted in alternative embodiments.) FIG. 1 is for delivery of one drug (in this case insulin) and shows the flow path from the reservoir to the patient in a straight line for simplicity. FIG. 1, 1) shows the reciprocating pump (100) in the neutral position prior to pumping. The pump's insulin dosing chamber (101) is adjacent to one side of the reciprocating pump (100). Insulin is stored in the insulin reservoir (102). Two active valves (103 and 104) are placed on either side of the dosing chamber, and the dispense confirmation senor (DCS) (105) is located in the flow path just prior to the dispense outlet to the patient (106). These components are controlled using a programmed control circuit that provides power to open and close the valves appropriately, provides the correct power to the pump to aspirate and dispense the proper amount of the therapeutic fluid, and records the flow signal from the DCS. At the conclusion of the dosing sequence, the controller analyzes the signal reported by the DCS and determines if the dose was given properly or if a dispense error occurred. The controller is capable of sending a successful dose completion signal or an error signal to an external controller that may contain a dosing algorithm.

Please note that in the neutral position 1) the active valve (104) closest to the patient is closed and therefore there is no direct flow path from the reservoir (102) to the patient (106), which is an important safety feature of the present invention. The patient is potentially exposed only to the minimal volume in the flow path up to the point of the active valve (104) should some significant pressure change or external force cause insulin to be expelled from the pump.

FIG. 1, 2) is the insulin dosing chamber fill step where the flexible walls of the reciprocating pump (100) are moved downward increasing the volume of the insulin dosing chamber (101) which results in aspiration of insulin from the insulin reservoir (102) through the open active valve (103) and into the insulin dosing chamber (101). Since active valve (104) is closed, no flow is detected by the DCS (105) and there is still no open fluidic path from the reservoir to the patient (106).

FIG. 1, 3) is the dispense step, where active valve (103) is closed and active valve (104) is opened. Note that in an intermediate step, both valves (103) and (104) are closed, so there is never an open fluidic path between the reservoir and the patient. The flexible walls of the reciprocating pump (100) are moved upward to decrease the volume of the insulin dosing chamber (101) which results in flow of insulin from the dosing chamber (101) through open active valve (104), past the DCS (105) [which indicates successful dispense] and into the patient (106). By repeating 2) and 3) the dosing chamber is refilled from the reservoir and a dose of insulin is given to the patient as needed. The outlet active valve (104) is closed any time the pump is not actively delivering insulin to the patient. Please note that in this first embodiment the bottom dosing chamber is not involved and only pushes back and forth against open air.

The rate of pumping is controlled so that the velocity of the therapeutic fluid in the flow path is such that the flow is laminar. This prevents fibrillation of medicaments containing hormones and also prevents shearing or other destruction of these and other therapeutic fluids that may result in loss of efficacy. The rate of flow can be controlled using a variety of design factors.

Without the active valve set (103 and 104), the reciprocating action of the pump would simply move the fluid back and forth; the addition of the set of active valves permits control of the direction of fluid movement. The two active valves (103 and 104) are operated in concert with the pump to minimize the opportunity for small variations in pumped volume to compound into larger variations as seen in other body-worn drug delivery pumps. Currently available syringe pumps have the entire reservoir (syringe) fluidically connected to the patient. Consequently, errors in a single dispense may affect the accuracy of subsequent dispenses or may have been affected by prior dispenses. In the present invention, dispense volume errors affect only the current dispense since the pump is separated from the reservoir during the pumping cycle by the closed valve (103). Additionally, the flexible diaphragm does not suffer from mechanical constraints such as gear tooth count and stiction between sliding parts that is present in syringe pumps. This improved precision of each reciprocating dispense translates into accuracy and precision of basal and bolus doses of all sizes, resulting in better overall accuracy during use.

During use and idle periods, a drug delivery pod may be subject to varying forces and pressures due to movement, changes in external pressure (e.g. during flight) or external contact on the pod while it is being worn (e.g. laying down). When the pump is not actively pumping, the valve (104) is always closed. Any external pressure on the pod or body will only be able to pressurize the small volume in the cannula and pod between the outlet and the closed valve (104) which will not have any effect on the pump and will not cause any movement of fluid within the system. The valve (104) is only open when the pump is actively pumping fluid to the outlet. In that case, having a pump that is capable of pumping against varying pressure, such as an electro-chemiosmotic pump, is required to ensure reliable dosing of the therapeutic fluid during operation.

Furthermore, drug delivery confirmation by the DCS increases the accuracy and safety by providing real time feedback on device operation, allowing the system to quickly detect any missed dispenses. The pressure-based occlusion detection method relies on detecting an increase in pressure of the outlet line between pump and patient. With the small dispense volumes required by some therapeutic fluids, the current sensors can take several hours to warn the patient they aren't receiving treatment. The flow-based sensor present in this first embodiment of the present invention detects the flow of solution thereby confirming success of, or reporting an error for each dispense. This high sensitivity prevents the pod from continuing to operate with repeated large under-dispenses. The DCS contributes to the overall safety of the drug delivery pod described here by notifying the user that they are not receiving the drug they need. In the case of diabetes this means the patient is able to take countermeasures, such as altering eating schedule or taking supplemental insulin injection to keep their blood glucose in range.

Finally, by alerting the user promptly, the DCS prevents the pod from developing a large outlet pressure which could be released to the patient in the case of a kinked infusion line. A pressurized outlet line can result in unintended dispense of insulin to the patient. (Insulin that should have been given over the course or several hours should not be released to the patient all at once.) This large overpressure is also alleviated by the active valves (103) and (104) in the fluid path. In case of occlusion near the patient, any increased pressure is relieved back to the reservoir (102) when the valve (103) is opened. However, the DCS (105) will still detect a lack of flow of drug to the patient (106).

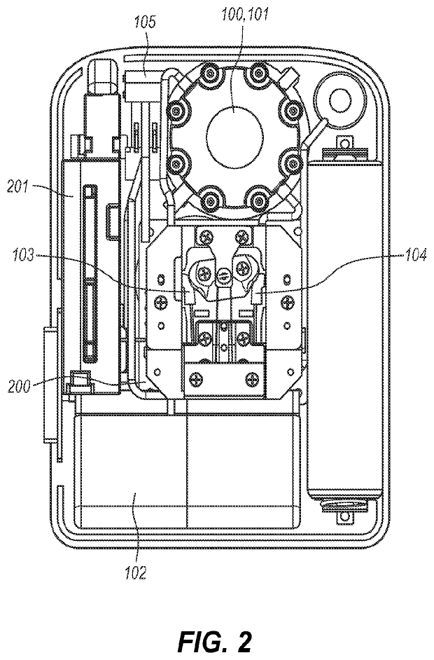

An insulin delivery pod according to a first embodiment of the invention is shown in FIG. 2. It includes the described enabling technologies: reciprocating pump (100) with dosing chamber (101), a set of active valves (103 and 104), and dispense confirmation sensor (105). It further includes standard technologies such as printed control circuit board (200), cannula inserter (201) and drug reservoir (102).

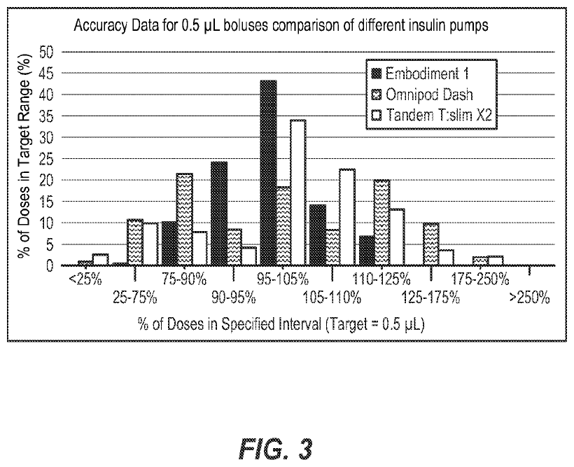

To demonstrate the low dosing capability of this first embodiment of the present invention, pods were programmed to dispense water (per IEC 60601-2-24 protocol) onto a balance at a rate of 5 μL /h (5 mg/h) given as one 0.5 μL (0.5 mg) bolus every 6 minutes. A total of five pods were tested, during which 2,400 doses were given. The results were compared to published results from similar tests performed using the FDA-cleared Omnipod® Dash® and Tandem Diabetes Care T:slim X2® (FIG. 3). FIG. 3 clearly shows that over 80% of the doses from the present invention were within ±10% of the targeted 0.5 μL (500 nL) dose, compared to only 35% of the doses from the Omnipod® device and 60% of the doses from the Tandem durable pump. Fewer than 1% of the doses from the present invention fell outside of the ±25% range whereas approximately 20% of the doses from the cleared delivery systems were off by more than a quarter of the dose (22% for the Omnipod® device and 18% for Tandem). This demonstrated accuracy corresponds to the present invention delivering a dose with an error of more than 25% less than once every 10 hours on average. (The comparative information came from FDA decision summary DEN180058 for T:slim X2® and from FDA decision summary K191679 for OmniPod® DASH®.)

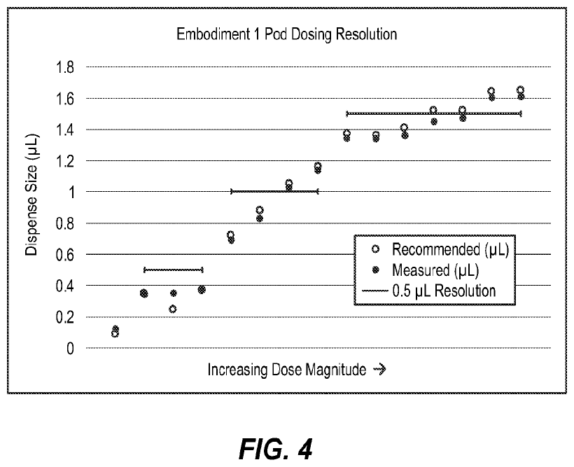

Another attribute of the present invention is resolution of dosing. It is important to note that the Omnipod® device can only deliver individual boluses in multiples of 0.5 μL due to mechanical limitations of the gears that drive the syringe. The present invention is not only more precise but can deliver boluses of any recommended dose. A dose of 0.6 μL or 0.55 μL, for example, would be delivered with similar precision as a 0.5 μL dose. In fact, the present invention was used to dispense doses onto a balance in response to an arbitrary artificial pancreas algorithm that recommended doses ranging from 0.1 μL to 2 μL with a resolution of 0.01 μL (10 nL) randomly. The overall accuracy error of this test was −2.0%, as shown in FIG. 4.

Since the second side of a reciprocating pump is also available to move fluid, a symmetric arrangement allows for the independent delivery of a second liquid medicament in a second embodiment of the present invention. This second embodiment could be used to deliver as needed any two fluid medicaments, especially those that would be delivered together frequently, such as insulin and glucagon, antibiotics and pain medications, HIV or cancer drug cocktails and more. Although not intended to be limiting, this discussion focuses on the independent, as-needed delivery of insulin and glucagon.

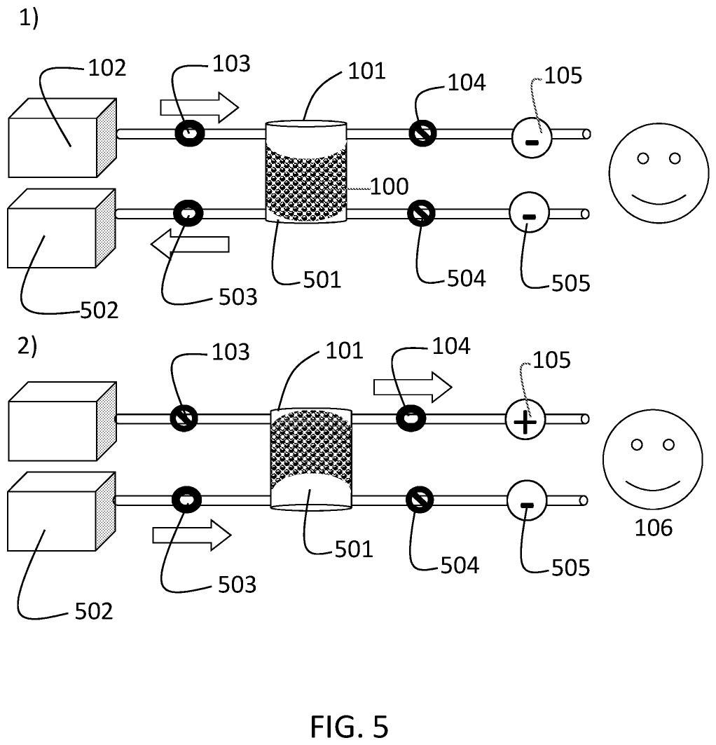

FIG. 5 shows how the second embodiment of the present invention delivers a dose of insulin without delivering a dose of glucagon and how the DCS is placed to confirm delivery of that dose of insulin as it is delivered through the cannula to the patient. Notice this second embodiment has two completely separate flow paths. In this description, the top flow path is for insulin only and the bottom flow path is for glucagon only. In FIG. 5, 1), the reciprocating pump (100) flexes downward to pull insulin into the insulin dosing chamber (101) from the insulin reservoir (102) through the open active valve (103). Since active valve (104) is closed there is no flow detected at the insulin dispense confirmation sensor (105). Simultaneously, the glucagon dose that was in the glucagon dosing chamber (501) of the reciprocating pump (100) is pushed back to the glucagon reservoir (502) through the open active valve (503). Since active valve (504) is closed there is no flow detected at the glucagon dispense confirmation sensor (505).

In FIG. 5, 2), the set of insulin active valves are switched such that active valve (103) is closed and active valve (104) is open. Note that in an intermediate state, both valves are closed, so there is never an open fluidic path between the reservoir (102) and the patient (106). The reciprocating pump (100) flexes upward to push insulin from the insulin dosing chamber (101) through the open active valve (104) past the insulin dispense confirmation senor (105) and into the patient (106). The signal provided by the insulin dispense confirmation sensor (105) confirms that the insulin dose has been delivered. Simultaneously, a glucagon dose is drawn from the glucagon reservoir (502) through open active valve (503) into the glucagon dosing chamber (501) where it is ready for delivery if needed or returned to the glucagon reservoir if not needed. The glucagon outlet valve (504) is never open to the patient during an insulin dispense. Multiple doses of insulin can be given to the patient without a single dose of glucagon being given by simply repeating FIGS. 5, 1) and 5, 2). Since the direction of flow through the flow path is controlled by the opening and closing of the valves, the pump is capable of bi-directional flow in the same system, such as would be required to return an unneeded dose back to the reservoir. Note that the only time the insulin outlet valve (104) is open and the insulin DCS (105) provides a delivery signal is when insulin is actually being delivered. Also note that at no time is there an open flow path between the reservoir and the patient for either the insulin flow path or the glucagon flow path because one of the two safety valves always remains closed in each set.

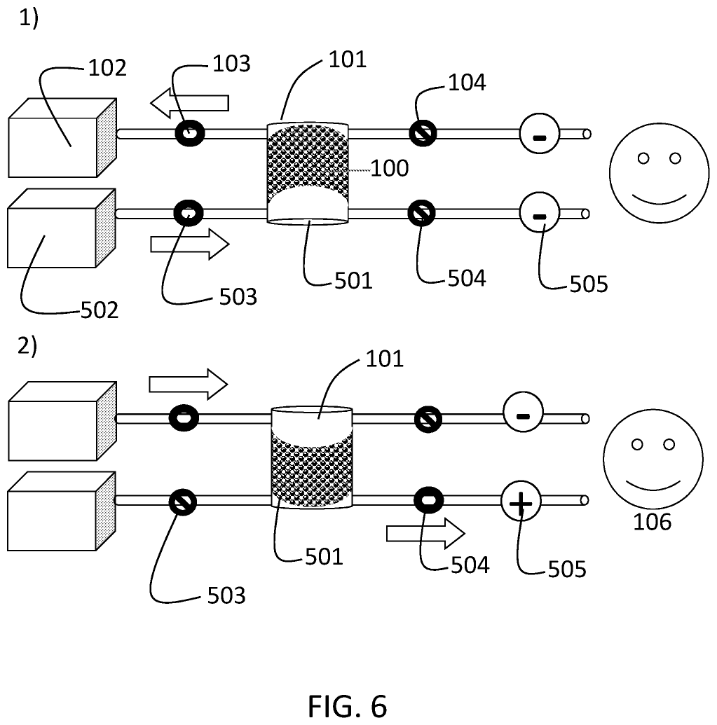

FIG. 6 shows how the second embodiment of the present invention delivers a dose of glucagon without delivering a dose of insulin and how the glucagon dispense confirmation sensor is placed to confirm delivery of that dose of glucagon as it is delivered through the cannula to the patient. Glucagon dosing is performed using similar actions to those described for FIG. 5. In this case, the insulin active valve (103) is open, and the insulin active valve (104) is closed for the duration of the glucagon dosing cycle. In FIG. 6, 1) the active valve (503) is open, and the active valve (504) is closed. The flexible walls of the reciprocating pump (100) flex upward so that the dose of glucagon is aspirated from the glucagon reservoir (502) to the glucagon dosing chamber (501). Simultaneously, a dose of insulin is returned from the insulin dosing chamber (101) through the open active valve (103) to the insulin reservoir (102). In FIG. 6, 2) inlet valve (503) is closed, and outlet valve (504) is opened. Note that in an intermediate step, both valves (503) and (504) are closed. Then the flexible walls of the reciprocating pump (100) flex downward and the glucagon dose is pushed through the open valve (504) and delivered to the patient (106). In this step, the glucagon dispense confirmation sensor (505) is showing confirmation of dose delivery.

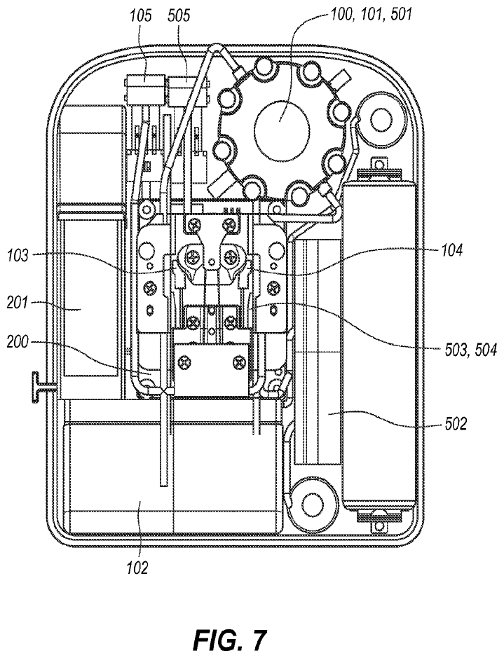

A second embodiment of the present invention for dual dispensing will be described as shown in FIG. 7. It includes the described technologies: reciprocating pump (100) with two dosing chambers (101 and 501), two sets of safety valves (103/104 and 503/504) stacked as mirror images of each other, and two dispense confirmation sensors (105 and 505); as well as more standard technologies such as a printed circuit board (PCB) (200), two cannula inserters (201), insulin reservoir (102), and a glucagon reservoir (502). This embodiment of the present invention uses two sets of safety valves on two separate flow paths to deliver each drug independently. Especially important to note is that the pod can deliver multiple doses of one drug without delivering any of the other.

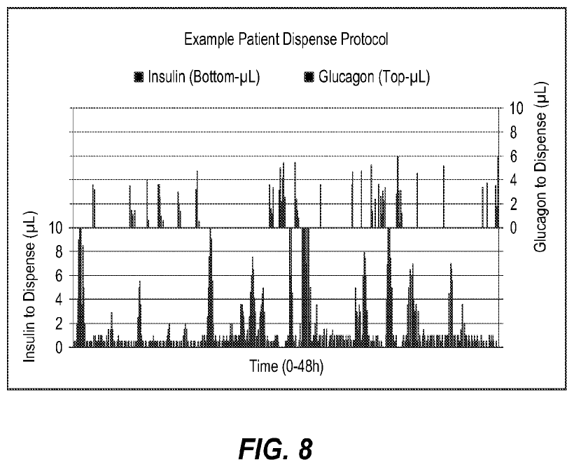

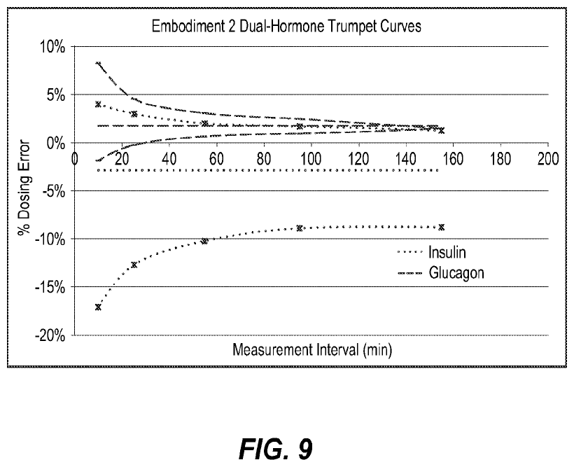

Since a reciprocating pump is the only pump that can independently deliver two drugs using a single pump, research to-date on dual hormone delivery uses two pumps: one for insulin and a separate one for glucagon. Insulin is used primarily to maintain glycemic control and glucagon is given less often to prevent or correct a predicted or existing hypoglycemic condition. One early trial used two pumps, one to deliver insulin and one to deliver glucagon in a trial of a Model Predictive Control dual-hormone algorithm developed by a research group led by Ed Damiano. From these published results, a patient was selected randomly, and the dosing protocol was repeated using the second embodiment of the present invention to deliver the recommended doses onto a two balance, dual-hormone dose measurement setup. The 48-hour dosing protocol is shown in FIG. 8, with dose recommendations given once every 5 minutes (some recommendations were 0 μL). With the dual-hormone apparatus, both drugs were accurate to within ±10% over any 1-hour window, and the average error for insulin was −2.8% or −0.016 U, and the average error for glucagon was +1.8% or +0.22 μg. The second embodiment of the present invention demonstrated accuracy much better than currently commercially available patch pumps. The trumpet curve in FIG. 9 summarizes the precision and accuracy of all of the doses given over the 48-hour period as described in the IEC 60601-2-24 protocol for quantification of dose accuracy and precision.

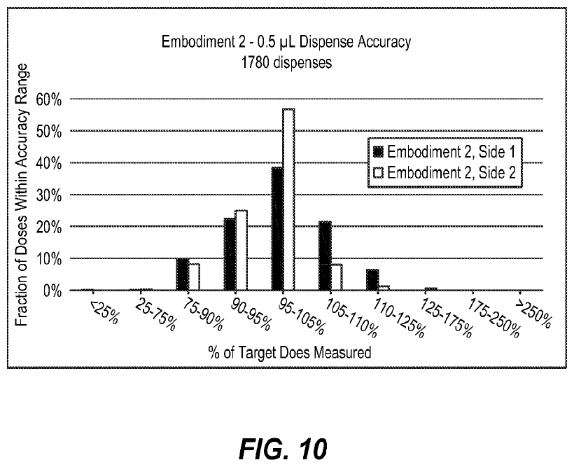

To demonstrate the low dosing capability of this second embodiment of the present invention, pod components were programmed to dispense water (per IEC 60601-2-24 protocol) at 0.5 μL (0.5 mg for water) per dispense alternately from each dosing chamber (101/501) of the ePump 100. This volume was chosen because it corresponds to the doses used to give a 5 μL/h dose rate using U100 from an existing insulin pump (0.5 μL/6 minutes). Four tests were performed, totaling 1780 doses. The results are shown in FIG. 10. Each side of the present invention dispenses onto a separate balance to ensure that there is no “crosstalk” between the two sides of the present invention.

FIG. 10 clearly shows that over 80% of the doses from either side of the second embodiment of the present invention were within ±10% of the targeted 0.5 μL dose. When compared to published data for the Omnipod® device and the Tandem pump, only 35% of the doses from the Omnipod® device and 60% of the doses from the Tandem pump fell in this range for this dispense volume. Fewer than 1% of the doses from the present invention fell outside of the ±25% range whereas approximately 20% of the doses from these other delivery systems were off by more than a quarter of the dose (22% for the Omnipod® device and 18% for Tandem). This corresponds to this second embodiment of the present invention delivering a dose with an error of more than 25% less than once every 10 hours on average. The total volume delivered over the course of the test was still well within ±10% of the target volume, even with a few individual dispenses being outside of this range. The comparative information here came from FDA decision summary DEN180058 for the Tandem T:slim X2® and from FDA decision summary K191679 for the OmniPod® DASH®. This discussion focuses on improved dispense accuracy/precision at more technically challenging small volumes. However, due to the reciprocating nature of the ePump, a large dose is simply the sum of several small doses and thus this embodiment of the present invention shows the same superior accuracy and precision at all clinically relevant dose volumes for both insulin and glucagon.

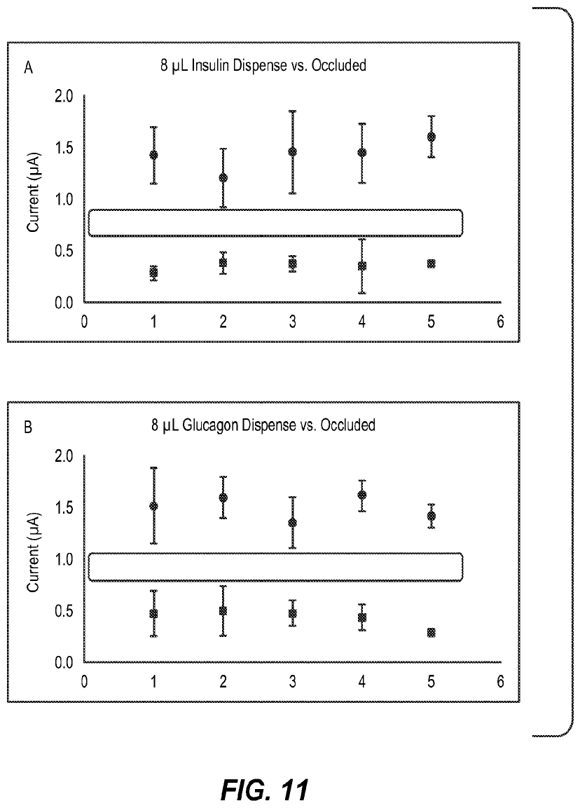

To demonstrate the ability of the present invention to detect occlusions during bolus dispensing, five DCSs were tested with multiple manually induced occlusions (per IEC 60601-2-24 protocol). Data demonstrating detection capabilities of insulin bolus sub-dispenses (8 μL) are shown from the present invention in FIG. 11A. The circles show the average current recorded by the DCS during an unobstructed insulin dispense in FIG. 11A. The average current recorded during pumping while occluded are shown as squares. Thirty-two occlusions were measured using five sensors. For both values, statistical error bars delineate the 2σ interval around each average current (approximately 95% confidence). A box illustrates the significant difference between average current during flow vs. the average current during occlusion. Likewise, for shelf-stable liquid glucagon formulation, the average current reported by the DCS during 8 μL dispense is shown as circles in FIG. 11B. Squares depict the average current recorded during occlusions. A total of 25 occlusions were spread over five sensors. Again, statistical error bars represent 2σ (95% confidence) interval around each data point. A threshold current value anywhere inside the box would discern which dispenses were delivered to the patient and which were not (dispense error). By averaging the current measured by the DCS and comparing to the threshold for the solution that was assigned to be administered, the present invention is easily able to confirm or deny that each dispense of either insulin or glucagon was administered properly to the patient. Compared to other occlusion detectors that can take many hours to sense blockage, the DCS is able to inform the control circuit about the status of each dispense before the next dispense. This permits the control circuit to alert the patient in a clinically relevant time frame. In addition to the onboard control circuit the confirmation of dispense, or lack of dispense (error) can be communicated to an algorithm that controls dispense of insulin or glucagon to the patient. This rapid feedback may permit the algorithm to take corrective action on its own without alerting the patient.

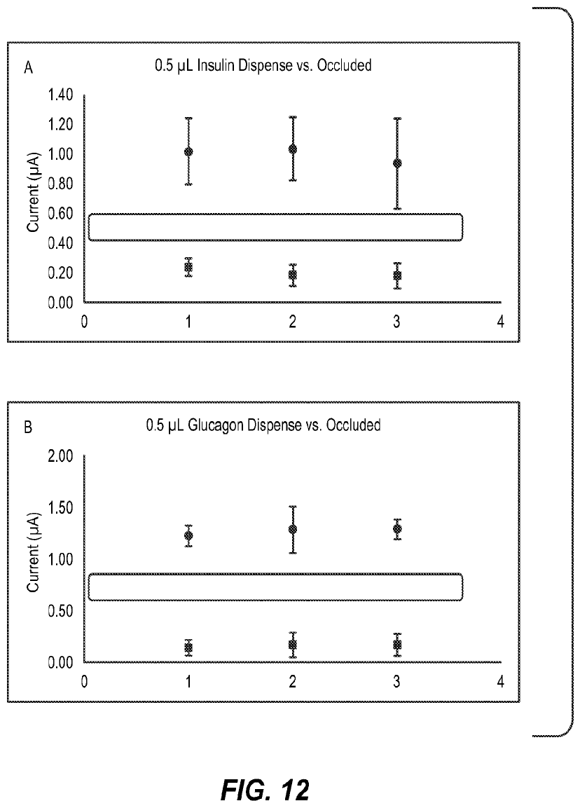

For confirmation of 0.5 μL dispenses, a pod like that used for the previous DCS tests was used to keep other aspects of testing the same. A total of three DCSs were used for testing individual 0.5 μL dispenses of insulin and glucagon solutions. For each DCS, multiple occlusions were manually induced by clamping soft tubing between the DCS and the balance. A restatement of data for the 0.5 μL testing performed for insulin delivery from the present invention is shown in FIG. 12A. FIG. 12B shows the similar data recorded for 0.5 μL doses of glucagon carrier solution. Again, for both charts, the circles depict the average current reported by the DCS during flow. Squares show the average current recorded when the outlet tube is manually occluded. A total of 64 occlusions were generated using three sensors for each fluid. Statistical error bars represent 2σ values. The box again delineates where an occlusion detection threshold could be placed for basal dispenses. Average current values above this threshold would be confirmed as having been delivered to the patient and average current values below this threshold would be confirmed as a dispense error, communicating that the dose had not been delivered to the patient. This data clearly indicates the ability of the present invention to “see” individual basal dispenses at an insulin basal rate of 5 μL/hr. or a micro-dose of 2 μg glucagon hormone.

In contrast to occlusion detection currently available on the market, the present invention has single dispense resolution. This sensitivity is vastly superior to other devices on the market and allows the device to alarm at clinically relevant dosing levels even for low body weight patients. For instance, a dual-hormone artificial pancreas system assumes the availability of glucagon to prevent or treat hypoglycemia. A simple use case of clinically relevant alert/alarm threshold can be based on continuous glucose monitoring (CGM) data. A low dose dispense of glucagon to correct projected hypoglycemia may only result in an alert notifying the patient if the present invention detects missed dispense. However, a missed dispense accompanying a low CGM reading may result in an alarm to the user, requiring the user to take corrective action.

With currently available insulin-only pods, the two main risks that exist due to occlusions are hyperglycemia resulting from occlusion of the insulin flow and hypoglycemia due to sudden release of accumulated insulin after an occlusion is cleared. Current testing protocols (IEC 60601-2-24, Section 201.12.4.4.104) require measurement of unintended dispense after detection and removing an occlusion. This protocol was followed for the occlusion testing conducted on this second embodiment of the present invention, except that a peristaltic valve was used to generate the occlusion instead of a stopcock. This adjustment was made due to the small diameter of the outlet tubing (1 mm OD, 0.5 mm ID) and the need for a small valve to generate the occlusion. Per IEC protocol, the tubing from the present invention dispensed onto a balance, so that the dispense volumes could be quantified by an increase in mass on the balance.

Some embodiments of the present invention use an ePump as the reciprocating pump and dual latching microvalves for the active valves set. Because ePump works in a reciprocating manner, a set of dual latching microvalves are used both to control the direction of flow of the fluid and to prevent a direct path from the reservoir to the outlet. In operation, the latching valve to the outlet is only open during the time that the pump is actively dispensing. For the remainder of the time, the outlet latching valve is closed and the inlet latching valve between the pump and reservoir is open. Since the valves are latching, power is only needed to change the state of the valves. No power is required to hold a closed latching valve in the closed position or an open latching valve in an open position. In the event of an occlusion, pressure generated in the pump is relieved back into the reservoir during the subsequent rest and fill portion of the cycle. Therefore, there is not continuous pressure on the patient side of the pump in the event of an occlusion and no unintended dispense will occur upon clearance of an occlusion.

The symmetric design of the ePump allows it to be assembled uniaxially. This is much easier for automated high-volume assembly processes. Uniaxial assembly will allow the pump to be made at a lower cost than other pumps that must be assembled to fit with the various gears and plungers that afford their operation. To support mass manufacture of the entire pumping system, including the flow paths, a fluidic manifold may be used to allow placement of each component into the pod easier and more reliable.

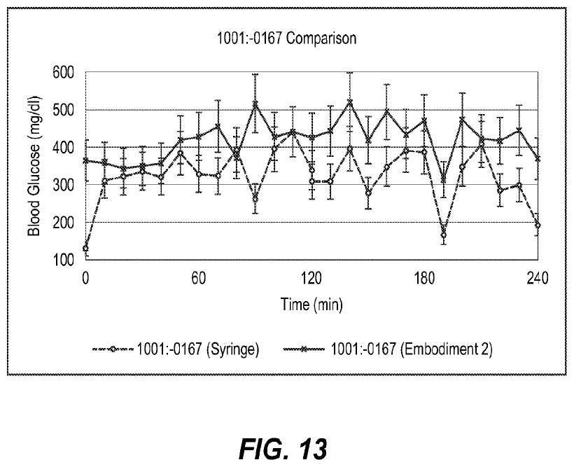

FIG. 13 shows the results of the second embodiment of the present invention being used in an in vivo test (diabetic swine model). In this test the present invention is being compared side-by-side to manual syringe injection of the same volumes of insulin and glucagon. A dose of insulin is given at t=0 and a dose of glucagon is given at t=120 minutes. FIG. 13 graphs the resultant blood glucose concentrations of syringe injection by hand vs automated injection vs the present invention. Though the blood glucose values are noisy, there is no significant difference between the change in blood glucose levels due to insulin and glucagon injection by hand vs. those delivered by the second embodiment of the present invention.

Unless otherwise stated, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It will be apparent to those skilled in the art that many more modifications are possible without departing from the inventive concepts herein.

All terms used herein should be interpreted in the broadest possible manner consistent with the context. When a grouping is used herein, all individual members of the group and all combinations and subcombinations possible of the group are intended to be individually included. When a range is stated herein, the range is intended to include all subranges and individual points within the range. All references cited herein are hereby incorporated by reference to the extent that there is no inconsistency with the disclosure of this specification.

The present invention has been described with reference to certain preferred and alternative embodiments that are intended to be exemplary only and not limiting to the full scope of the present invention, as set forth in the appended claims.

1. A fluid delivery device, comprising:
a reciprocating pump positioned in a one flow path between a reservoir and an outlet, wherein the reciprocating pump comprises a first side;
a dosing chamber adjacent to the first side of the reciprocating pump;
a first active valve positioned in the first flow path between the reservoir and the dosing chamber;
a second active valve positioned between the dosing chamber and the outlet;
a control circuit configured to independently control the pump, the first active valve, and the second active valve to control flow from the reservoir to the outlet.

2. The fluid delivery device of claim 1, wherein a target dose volume of the therapeutic fluid is in the range of 1 μL to 3 mL.

3. The fluid delivery device of claim 2, wherein ninety percent of the target dose volumes are delivered with an accuracy in the range of plus or minus ten percent.

4. The fluid delivery device of claim 1, wherein a target dose volume of the therapeutic fluid is in the range of 1 nL to 1 μL.

5. The fluid delivery device of claim 4, wherein eighty percent of the target dose volumes are delivered with an accuracy in the range of plus or minus ten percent.

6. The fluid delivery device of claim 1, wherein the target dose volumes are delivered with a resolution of 10 nL.

7. The fluid delivery device of claim 1, wherein the dose is delivered in laminar flow conditions wherein the therapeutic fluid remains efficacious between the reservoir and the outlet.

8. The fluid delivery device of claim 1, wherein operation remains reliable when operating against varying pressure.

9. The fluid delivery device of claim 1, wherein the first active valve and the second active valve are a set of dual latching microvalves.

10. The fluid delivery device of claim 1, wherein the first active valve remains in an open position or a closed position when no power is applied from the control circuit.

11. The fluid delivery device of claim 1, wherein the second active valve remains in the open position or the closed position when no power is applied from the control circuit.

12. The fluid delivery device of claim 1, wherein the reciprocating pump is assembled uniaxially.

13. The fluid delivery device of claim 1, wherein a method of operation for the reciprocating pump is selected from the group consisting of electrochemical, electroosmotic, and electrochemiosmotic.

14. The fluid delivery device of claim 1 wherein the first and second active valves each comprise insert molded spring arms.

15. The fluid delivery device of claim 1 wherein the fluidic path contains or is contained in a fluidic manifold.

16. The fluid delivery device of claim 1 wherein the reciprocating pump further comprises a second side opposite to the first side, and a second dosing chamber adjacent to the second side of the reciprocating pump.

17. The fluid delivery device of claim 16 wherein the second dosing chamber is connected in a second flow path with a second reservoir and a second valve set to deliver a complimentary therapeutic fluid to an outlet of the second flow path.

18. The fluid delivery device of claim 17 where the first dosing chamber can dispense one or more doses of a first therapeutic fluid without the second dosing chamber dispensing any therapeutic fluid.

19. The fluid delivery device of claim 18, wherein the control circuit is configured to control bi-directional flow from the reciprocating pump.

20. A fluid delivery device, comprising:
a reciprocating pump positioned in a flow path;
a first active valve positioned in the flow path between a reservoir and the reciprocating pump;
a second active valve positioned between the reciprocating pump and an outlet of the flow path;
a first dispense confirmation sensor in the flow path between the second active valve and the outlet of the flow path configured to send a signal to the control circuit indicative of at least one of a dispense and a dispense error, wherein the dispense error may comprise one or more of an occlusion in the flow path, an empty reservoir, and a failure of the reciprocating pump; and
a control circuit configured to independently control the pump, the first active valve, and second active valve to control flow from the reservoir to the output of the flow path and to receive the signal from the first dispense confirmation sensor.

21. The fluid delivery device of claim 20, wherein the first dispense confirmation sensor is configured to send either a confirmation or a dispense error message to the control circuit prior to a next dispense.

22. The fluid delivery device of claim 20, wherein the control circuit is further configured to dispense a first therapeutic fluid according to a dosing algorithm, and wherein the control circuit is further configured to receive the signal as a dispense signal from the first dispense confirmation sensor to inform the dosing algorithm.

23. The fluid delivery device of claim 22, wherein the control circuit is further configured to receive the signal as a dispense error signal from the first dispense confirmation sensor to inform the dosing algorithm.

24. The fluid delivery device of claim 20, wherein the control circuit is configured to take the signal from the first dispense confirmation sensor as input to algorithmically determine dispense or error conditions.

25. The fluid delivery device of claim 20, wherein the sensitivity of the control circuit to report a dispense error to the patient is based on clinical relevance.

26. The fluid delivery device of claim 20, wherein the control circuit compares the average current to a set threshold to confirm dispense or error in dispense.

27. The fluid delivery device of claim 20, further comprising a second dispense confirmation sensor to confirm delivery of a second therapeutic fluid.

28. The fluid delivery device of claim 27, wherein the sensitivity of the control circuit to report a dispense error to the patient is based on clinical relevance.

29. A method of delivering a fluid to a patient, comprising the steps of:
positioning a reciprocating pump between a reservoir and an outlet to create a single flow path;
positioning a first active valve between the reservoir and the reciprocating pump;
positioning a second active valve between the reciprocating pump and the outlet;
opening the first active valve to allow the flow of a dose of therapeutic fluid between the reservoir and the pump;
closing the first active valve;
opening the second active valve to allow the dose of therapeutic fluid to flow from the outlet; and
closing the second active valve.

30. The method of claim 29, wherein the opening and closing steps are repeated to deliver multiple doses of therapeutic fluid.

31. The method of claim 29, wherein the target dose volume of the fluid is in the range of 1 nL to 3 mL.

32. The method of claim 31, wherein eighty percent of the target dose volumes are delivered with an accuracy in the range of plus or minus ten percent.

33. The method of claim 29, further comprising the step of creating a second independently controlled flow path on a second side of the reciprocating pump to allow for as needed delivery of a second therapeutic fluid.

34. The method of claim 29, further comprising the step of confirming a dispense or dispense error with a dispense confirmation sensor between the second active valve and the outlet

35. The method of claim 34, further comprising the step of relaying the confirmation or dispense error to a control system to inform the user or a dosing algorithm.
